SEQUENCE LISTING (9 sequences)

SEQ ID NO: 1 - DNA, 41 bp, synthetic construct
  source 1..41: mol_type = other DNA
gacaggctgtggggtttctctcccgggactctactaccttt

SEQ ID NO: 2 - DNA, 40 bp, synthetic construct
  source 1..40: mol_type = other DNA
gtaattcccgcgcttttccgtctgtccctcccatcctctg

SEQ ID NO: 3 - DNA, 20 bp, synthetic construct
  source 1..20: mol_type = other DNA
gaaatccactctcccacgcc

SEQ ID NO: 4 - DNA, 20 bp, synthetic construct
  source 1..20: mol_type = other DNA
caatccagagccccgagaga

SEQ ID NO: 5 - DNA, 19 bp, synthetic construct
  source 1..19: mol_type = other DNA
tcaggaggccttcaccctc

SEQ ID NO: 6 - DNA, 19 bp, synthetic construct
  source 1..19: mol_type = other DNA
ggaaaccaaggggctaccg

SEQ ID NO: 7 - DNA, 2001 bp, Homo sapiens
  source 1..2001: mol_type = other DNA
ctcttgtgctgacttaccagatgggacactctaagattttctgcatagcattaatgacattttgtacttcttcaacgcgaagagcagataaatccatttctttctgttccaatgaactttaacacattagaaaaacatatatatatatctttttaaaaggtttataaaatgacaacttcattttatcattttaaaataaagtaaatttaagatttggaaggttttagaataatacaaaccaaagaactaatgacaacgtcctttatttttaaagattctagaagttgctttttgtaattagacaacataaattctgaattttttcacatattgctgccaaccccttgggtcttttcctttctccaagaaagagaaagctacagaggagtgactgaccgggtaggtggtggtagccttagctttctccaatgtttctggttgttttctttttcttgcataaaaccaaaatcaacaacgaccaaaccaacaccaatcaaggcctccccgcccctaacctttcccagtgacctgctctcatctctggatcctcctcaagcacatccctgccggcagcatctgttactactgacgctcctctacttccctcttgcgctttctcaatggcgcaaatggatccagttcttaagttctccctcccacaaaatcctgtctcctccccttcccagacatattcctggcacctcttcttccacaaggtcccatcctctcatacataccagccggtgttttttgttttgttttgttttgttttgttttgagacagtctcgctctgtcgcccaggctggagtgcaatggcgcgatctcggctcactgcaacctccgcctcccgggttctagcgattctcctgcctcagcctcctgagtagctggagcggcaccacgcccggctaatttttgtatttttagtagagacggagtttcaccacgttggtcaggctggtctggaactcctgacctcatgaccagccgacgtttttaaagacatagtgtccccctcaaggcatattccagttcctatcacgaggattcccccacggacactcagtgcccccttcctgatcctcagcgcttccctcgcgacctacaaactgcccccctccccagggttcacaacgccttacgcctctcaggttccgcccctaccccccgtcaaagaatacccatctgtcagcttcggaaatccactctcccacgccagtaccccagagcatcacttgggccccctgtccctttcccgggactctactacctttacccagagcagagggtgaaggcctcctgagcgcaggggcccagttatctgagaaaccccacagcctgtcccccgtccaggaagtctcagcgagctcacgccgcgcagtcgcagttttaatttatctgtaattcccgcgcttttccgttgccacggaaaccaaggggctaccgctaagcagcagcctctcagaatacgaaatcaaggtacaatcagaggatgggagggacagaaagagccaagcgtctctcggggctctggattggccacccagtctgcccccggatgacgtaaaaggaaagagacggaagaggaagaattctacctgagtttgccataaagtgcctgccctctagcctctactcttccagttgcggcttattgcatcacagtaattgctgtacgaaggtcagaatcgctacctattgtccaaagcagtcgtaagaagaggtcccaatcccccactctttccgccctaatggaggtctccagtttcggtaaatataagtaataaggattgttgggggggtggagggaaataattatttccagcatgcgttgcggaatgaaaggtcttcgccacagtgttccttagaaactgtagtcttatggagaggaacatccaataccagagcgggcacaattctcacggaaatccagtggatagattggagacctgtgcgcgcttgtacttgtcaacagttatggactggagtgttatgttttc

SEQ ID NO: 8 - DNA, 19 bp, synthetic construct
  source 1..19: mol_type = other DNA
tcaggaggccttcaccctc

SEQ ID NO: 9 - DNA, 19 bp, synthetic construct
  source 1..19: mol_type = other DNA
ggaaaccaaggggctaccg

(19) United States
(12) Patent Application Publication
(10) Pub. No.: US 2026/0347915 A1
(43) Pub. Date: Nov. 5, 2026

(54) CORE-SHELL HYDROGEL PARTICLES WITH TUNABLE POROSITY FOR DIGITAL NUCLEIC ACID ASSAYS

(71) Applicant: THE REGENTS OF THE UNIVERSITY OF CALIFORNIA, Oakland, CA (US)

(72) Inventors: Dino Di Carlo, Los Angeles, CA (US); Michael Thomas Alesse Bogumil, Northridge, CA (US)

(73) Assignee: THE REGENTS OF THE UNIVERSITY OF CALIFORNIA, Oakland, CA (US)

(21) Appl. No.: 19/115,238

(22) PCT Filed: Oct. 5, 2023
(86) PCT No.: PCT/US2023/076150

§ 371 Date: Mar. 25, 2025

Related U.S. Application Data
(60) Provisional application No. 63/378,655, filed on Oct. 6, 2022.

(51) Int. Cl. C12Q 1/6844

(52) U.S. Cl. CPC C12Q 1/6844

(57) ABSTRACT

A core-shell particle system for performing nucleic acid amplification of targets includes particles having a hydrogel outer shell surrounding a core region that is substantially devoid of hydrogel, wherein the outer shell comprises pores having a size that permit the passage of the targets and nucleic acid reagents into the core region from an external environment surrounding the particles while substantially preventing the escape of amplicons generated in the core region in response to nucleic acid amplification. The system is used by mixing the particles with target and nucleic acid amplification reagents and partitioning the particles from one another. The particles are incubated for a period of time so as to generate amplicons. The particles are then de-partitioned from one another. The de-partitioned particles are then exposed to a dye or fluorescent reporter that is specific to nucleic acids or to a target amplicon and optically interrogated.

### RELATED APPLICATION

This application claims priority to U.S. Provisional Patent Application No. 63/378,655 filed on Oct. 6, 2023, which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number 1648451, awarded by National Science Foundation. The government has certain rights in the invention.

### TECHNICAL FIELD

The technical field generally relates to microscale compartments with molecular weight cutoffs and methods of using the same in conjunction with nucleic acid assays. More specifically, the technical field pertains to hydrogel-based core-shell particles having tunable porosities. The particles are used as reaction vessels to generate higher molecular weight reporter molecules that are trapped within the core of the particles in response to the presence of target molecules. The reporter molecules can be interrogated optically using for example, flow cytometry or microscopy.

### BACKGROUND

Quantifying nucleic acid concentration has been an extremely important tool for biological research and diagnostics. The 2020 pandemic taught the importance of having low-cost readily available Nucleic Acid Amplification Tests (NAATs) for screening and diagnostics. By quantifying the SARS-CoV-2 RNA concentration in a patient sample the elucidated viral copy number may be detected and infection diagnosed. This is true in many types of infectious diseases in which viral or bacterial genetic information is used to diagnose infection from various sample types, including blood, urine, nasal swabs, saliva, tears, etc. Beyond infectious diseases, NAATs can be used as cancer diagnostics by quantifying circulating cell-free DNA (cfDNA) or specific methylated cfDNA concentrations in patient blood samples. NAATs have also been used for public health application such as to measure the concentration of pathogens in drinking water or monitor outbreaks by measuring pathogen concentration in wastewater.

Nucleic acid quantification is mostly performed using bulk assays such as qPCR or even with Next-Generation sequencing (NGS). NGS can be prohibitively expensive, and qPCR suffers from accuracy and precision issues, necessitating the need to simultaneously run a standard calibration curve with an assay. In the last couple of decades, the rise of digital nucleic acid amplification tests (dNAATs) has remedied many of the issues with traditional bulk assays.

Digital assays work by breaking up an assay solution into many small discrete volumes. By separating the test solution into a number partitions, the analyte can be probed on a single-molecule level. The nucleic acid target molecules can then be amplified inside of the partitions in the presence of an intercalating dye or other sequence specific nucleic acid probe (e.g., TaqMan® probes from ThermoFisher Scientific). The partition can then be analyzed by measuring the fluorescence or other colorimetric, optical or electrochemical read out method. Partitions that are fluorescent above a threshold contained one or more targets while partitions that are not fluorescent above a threshold value did not contain any target. The ratio of these two populations can then be used to calculate a target concentration using Poisson loading statistics.

Partitioning creates an increase in the nucleic acid target concentration in an individual partition, decreasing the limit of detection (LOD) of the assay, making it more sensitive. Partitioning also enriches low-abundance targets in comparison to other contaminating nucleic acids which can be vital for cancer diagnostics, in which wild type background genes may be present. The dynamic range of a digital assays can be extended by increasing the number of partitions generated. Since dNAATs interrogate targets on the discrete molecular level, it is able to absolutely quantify the target concentration. This precludes the need to create a standard curve, reducing the reagent use and increasing the usability.

Currently there are a couple partitioning technologies that enable dNAATs. The simplest of these is the common lab well plate. Due to the number of wells and volume of the wells the dynamic range is severely limited. The difficulty of a well plates' large partition volume is solved with microwells such as Thermofisher's Combinati system. This system suffers from the fixed number of wells on each chip. This limits the lower end of the dynamic range of the system making it difficult to measure lower concentrations or rare targets. BioRad QX200 droplet digital PCR system meets this challenge by partitioning the assay using droplets in oil. The number of droplets can be increased for rare targets. The BioRad and Thermofisher systems, however, both require expensive and specialized hardware to run their assays.

### SUMMARY

In one embodiment, the microscale compartments are PEG-based core-shell hydrogel microparticles that can easily create uniform partitions enabling users to perform dNAATs and counting of fluorescent partitions with commercial flow cytometers, which are present in most research laboratories and core facilities. In one embodiment, the sensitivity of Loop-mediated Isothermal Amplification (LAMP) is leveraged to amplify and detect targets of interest. By modulating the particle's shell porosity, the high-molecular weight DNA concatemers generated by LAMP amplification (i.e., amplicons) remain trapped in the particles for easy downstream analysis in a surrounding aqueous phase. While LAMP is specifically illustrated, it should be appreciated that other nucleic acid amplification techniques may also be used with the core-shell hydrogel particles.

In one embodiment, porous microscale compartments with a molecular weight cutoff are mixed with an aqueous sample and reagent mix. Target molecules from the sample pass into the porous compartments along with reagents that are used to create a higher molecular weight reporter molecule in the presence of target molecules. Higher molecular weight reporter molecules remain entrapped in the porous compartments and detected. Sample and reagents can then transport out of compartments, leading to selective signal where target molecules are present in microscale compartments. A plurality of microscale compartments or microparticles can be analyzed to provide a count of the number of target molecules in the sample. Microscale compartments can be analyzed using various detection modalities, including flow cytometry and microscopy.

In one embodiment, microscale compartments comprise hydrogel shell particles with a porous shell and hollow interior region. Shell particles are fabricated to have a molecular weight or size cutoff of the porous outer hydrogel shell above that of a target nucleic acid molecular weight (or size cutoff) and the molecular weight (or size cutoff) of a polymerase enzyme. Thousands to millions of shell particles are mixed with a sample containing target nucleic acids and a polymerase reagent mix and primers to perform loop-mediated isothermal amplification (LAMP) and allowed to incubate such that target nucleic acids and other reagents for LAMP transport into the interior region of the shell particles. The sample mixed with shell particles is then introduced to an oil phase with surfactant and mixed to emulsify and create drops in an external oil phase, where substantially all of the drops each contain a single shell particle. The mixture is incubated at elevated temperature to perform the LAMP reaction. The emulsion is then broken and shell particles are collected back into an aqueous phase. Unbound nucleic acids and other reagents are washed from the shell particles. Amplicons from LAMP remain entrapped in some shell particles that had target nucleic acids present. Amplicons are selectively labeled with fluorescent intercalating dyes, or target specific nucleic acid probes that hybridize and contain fluorophores. The number of shell particles with a fluorescence signal above a threshold are detected and analyzed to report out qualitative or quantitative information regarding the sample. This may include, for example, detecting that the sample was positive (+) for the target nucleic acid. This may also include a concentration of the target nucleic acid. Detection can be performed using a fluorescence microscope or flow cytometer or other optical imaging instrument. Shell particles can also be labeled with fluorophores to ease detection by microscopy and flow cytometry and detect coincident fluorescence signal of a shell particle and entrapped amplicon. For example, gating for shell particles above a fluorescence threshold in a first fluorescence channel, and target-positive particles above a threshold in a second fluorescence channel. In some embodiments shell particles are analyzed by a fluorescence activated cell sorter and shell particles with signal above a threshold are sorted for downstream molecular or genetic analysis. Genetic analysis may include analysis of mutations in a target nucleic acid sequence.

In one embodiment, a core-shell particle system for performing amplification of a target nucleic acid includes a plurality of particles comprising a hydrogel outer shell surrounding a core region that is substantially devoid of hydrogel, wherein the hydrogel outer shell comprises pores having a size that permit the passage of the target nucleic acid and nucleic acid amplification reagents into the core region from an external environment surrounding the plurality of particles while substantially preventing the escape of amplicons generated in the core region in response to nucleic acid amplification of the target nucleic acid.

In one embodiment, the particles may have a diameter within the range of 10 μm to 300 μm. The thickness of the outer shell of the particles may vary, for example, the outer shell may have a thickness within the range of 0.5 μm to 100 μm. In some embodiments, the plurality of particles may be conjugated to a light emitting reporter or dye which is used to better identify particles. The pore size of a particular particle system may vary. The pore size can be used to generate a size cut-off whereby molecules sized above the cut-off are substantially prevented from traversing the outer shell of the particles. Conversely, molecules sized below the cut-off are able to traverse the outer shell of the particles. In one embodiment, the pores have a molecular weight cut-off (MWCO) within the range of 50 kDa to 1 MDa. In another embodiment, the pores have a base pair length cutoff within the range of 200 bps (0.2 kbps) to 1200 bps (1.2 kbps). The target nucleic acid may include, for example, cfDNA, gDNA, eDNA, cDNA, DNA digested or broken into smaller length units using enzymes such as restriction enzymes, or viral subunits.

The particles, in some embodiments, are initially exposed to a sample in an aqueous environment which is followed by partitioning particles from one another with an oil phase. In another embodiment, the particles are partitioned from one another with an aqueous phase containing a high-molecular weight polysaccharide. In another embodiment, the particles are partitioned from one another with an aqueous phase containing a target nucleic acid chelator.

In one embodiment, the plurality of particles include poly(N-isopropylacrylamide) (PNIPAM) in the outer shell. In some embodiments, the plurality of particles have oligonucleotides conjugated to the outer shells within the core regions of the particles. In another embodiment, the core regions of the plurality of particles comprises a positively charged polymer contained therein.

In another embodiment, a method of using the core-shell particle system includes the operations of: mixing the plurality of particles with a sample containing the target nucleic acid and nucleic acid amplification reagents; partitioning the plurality of particles from one another; incubating the plurality of particles for a period of time so as to generate target amplicons; de-partitioning the plurality of particles from one another; exposing the de-partitioned plurality of particles to a dye or fluorescent reporter that labels nucleic acids or is specific to a target amplicon; and optically interrogating the plurality of particles.

The optical interrogation of the plurality of particles may be performed using, for example, a microscope or other optical imaging device, a flow cytometer, or a fluorescence activated cell sorter.

After optical interrogation of the plurality of particles, in one embodiment, the particles are binned into two groups, namely, particles that exhibit a positive (+) signal or particles that exhibit a negative (−) signal. In another embodiment, a concentration of the target nucleic acid is then performed based at least in part on the fraction of particles that exhibit a positive or negative signal above or below a threshold in response to optical interrogation. The particles may be conjugated with a different light emitting reporter that emits light in a different wavelength or wavelength range as compared to the dye or fluorescent reporter that labels nucleic acids or is specific to a target amplicon.

In one embodiment, prior to optical interrogation, the particles are exposed to a solution of differing ionic strength to reduce the size of the plurality of particles.

A variety of different nucleic amplification reactions may be used with the particles. Examples include rolling circle amplification (RCA), loop-mediated isothermal amplification (LAMP), or polymerase chain reaction (PCR).

### BRIEF DESCRIPTION OF THE DRAWINGS

FIG. 1 illustrates a core-shell particle exposed to a sample containing a target nucleic acid. The target nucleic acid is able to traverse across the outer shell of the particle and is amplified by an amplification reaction that takes place in the core region of the particle. The generated amplicons are not able to substantially traverse the outer shell and remain confined in the core region of the particle.

FIG. 2 schematically illustrates how the particles may be modified. Due to the use of dithiothreitol as a crosslinker there are many free thiol groups that can be used to modify the hydrogel material that forms the shell after it has been crosslinked. Species such as DNA probes, dyes and other species that could add extra functionality to the hydrogel can be conjugated to the particles. The species can be functionalized with maleimide chemistries to perform easy conjugation to the hydrogel. To prevent interference of any remain thiols in down-stream processes, the thiols can be inactivated by reacting with N-ethylmaleimide.

FIG. 3 schematically illustrates how core-shell particles are fabricated. The particles are fabricated by coflowing two precursor solutions in to flow-focusing droplet generator. The components of the two precursors form an aqueous two-phase system that separated to form a core-shell morphology. The shell is crosslinked with UV and then the particles are collected. The droplet generation oil is then removed, and the particles are washed in PBS to remove the dextran in the core. This results in the core-shell particles that can be used for digital assays.

FIG. 4 illustrates an illustrative workflow for dNAAT with the core-shell particles. The particles are mixed with the amplification buffer, nucleic acid target and any primers that might be needed. The target then diffuses into the particles. The particles are then partitioned from one another to prevent transport of the target or its amplification product once amplification has begun. The particles are then incubated to initiate amplification, after which the particles are de-partitioned. The de-partitioned particles are then washed with an intercalation dye to stain the captured DNA in the positive partitions. The particles can then be analyzed through flow cytometric, microscopy, or other methods.

FIGS. 5A and 5B illustrate how pore size may be tuned or adjusted with porogens. A DNA ladder and gel electrophoresis were used to characterize the porosity of core-shell particles with varying relative porogen concentrations. With reference to FIG. 5A, as the porogen concentration increases, larger DNA bands of the ladder are allowed to diffuse across the shell into the center, indicating larger pore sizes. With reference to FIG. 5B, through quantification of the gel electrophoresis images, it can be seen that Pluronic F127 can be used as a porogen to modulate pore size and molecular weight cut-off (★) of the particles across almost an entire order of magnitude, from 200 bps to 1200 bps.

FIGS. 6A-6C illustrate a DNA sweep for the dLAMP assay on the Sony SH800. To measure sensitivity on the Sony SH800 a sweep across varying concentrations of the BRCA1 target were performed. The target concentrations used in final particle mix were: 0, 4.9e4, 4.9e5, 4.9e6, 4.9e7 copies/μL. The gating used to separate the total particle and positive particle populations can be seen in FIG. 6A. Sample microscopic images of each condition were also taken and shown in FIG. 6B. FIG. 6C shows each condition plotted with the prepared DNA target concentration compared against its measured target per partition concentration.

FIGS. 7A-7C illustrate a DNA sweep for the dLAMP assay on the On-Chip Sort fluorescence activated cell sorter. To measure sensitivity on the On-Chip a sweep across varying concentrations of the BRCA1 target were performed. The target concentrations used in final particle mix were: 0, 4.9e5, 4.9e7 copies/μL. The gating used to separate the total particle and positive particle populations can be seen in FIG. 7A. Sample microscopic images of each condition were also taken and shown in FIG. 7B. FIG. 7C shows each condition plotted with the prepared DNA target concentration compared against its measured target per partition concentration.

FIG. 8 illustrates modulating core size in core-shell particles though the Aqueous Two-Phase System (ATPS). The core of the core-shell particles can be modified by changing the relative concentration the components of the ATPS. The data show a binodal curve indicating an interface in which an ATPS composed of 4-arm 10 kDa PEG-Norbornene and 40 kDa Dextran will phase separate. Above the binodal curve the system exists as two phases, below it exists as one phase. The maximum PEG and Dextran concentration are limited by their solubility. In between their solubility line and binodal curve, the relative concentration can be adjusted to modify the ratio if inner radius to outer radius of the core-shell particles. This can be seen in the bold contour lines.

FIG. 9 shows an example of particle size modulation using ionic strength of a storage solution. Here, the size of the fabricated core-shell particles can be modulated by placing them in solution of differing ionic strengths. By placing the particles in 5M NaCl the particles can be shrunk down by half their size, or shrunk down by almost 10-fold in volume.

### DETAILED DESCRIPTION OF ILLUSTRATED EMBODIMENTS

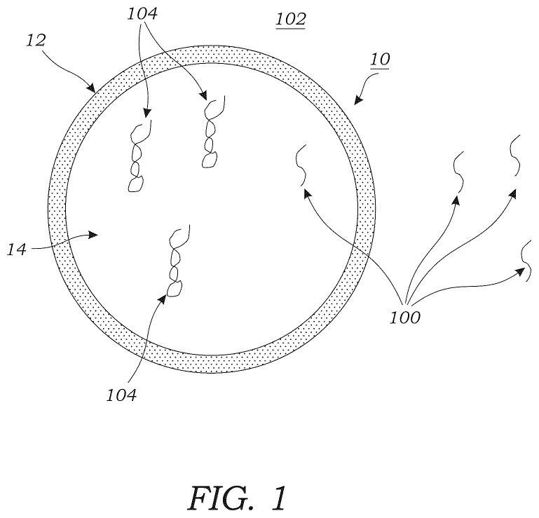

FIG. 1 illustrates a core-shell particle 10 for performing amplification of a target nucleic acid 100 present within a sample 102. The core-shell particle 10 has a hydrogel outer shell 12 that surrounds a core region 14 that is substantially devoid of hydrogel. The size of the core-shell particles 10 may vary but is typically between 10 μm and 300 μm in diameter and more preferably between 30 μm and 100 μm in diameter. The core-shell particles 10 are composed of a hydrogel shell 12 that allows nucleic acid target 100 and other analytes required for the amplification process to diffuse through the shell 12 and into the core region 14 that is substantially devoid of polymer. The thickness of the shell 12 is preferably between 0.5 μm to 100 μm. The porosity of the shell 12 can be tuned for a pore size ranging from 50 kDa to 1 MDa. In this regard, pore size is quantified as the molecular weight cut-off in which molecular weights below the cut-off value are able to traverse the pores of the shell 12 while molecular weights above the cut-off value are not able to traverse the pores of the shell 12. The porosity of the shell 12 may also be expressed in terms of base pair length. In this context, the pores, in one embodiment, have a base pair length cutoff within the range of 200 bps to 1200 bps. The particular pore size or size range that is present in the shell 12 defines a cut-off that allows target nucleic acids 100 and other reagents to enter the core-shell particle 10 from the external environment while at the same time substantially preventing the escape of amplicons 104 generated in the core region 14 in response to nucleic acid amplification.

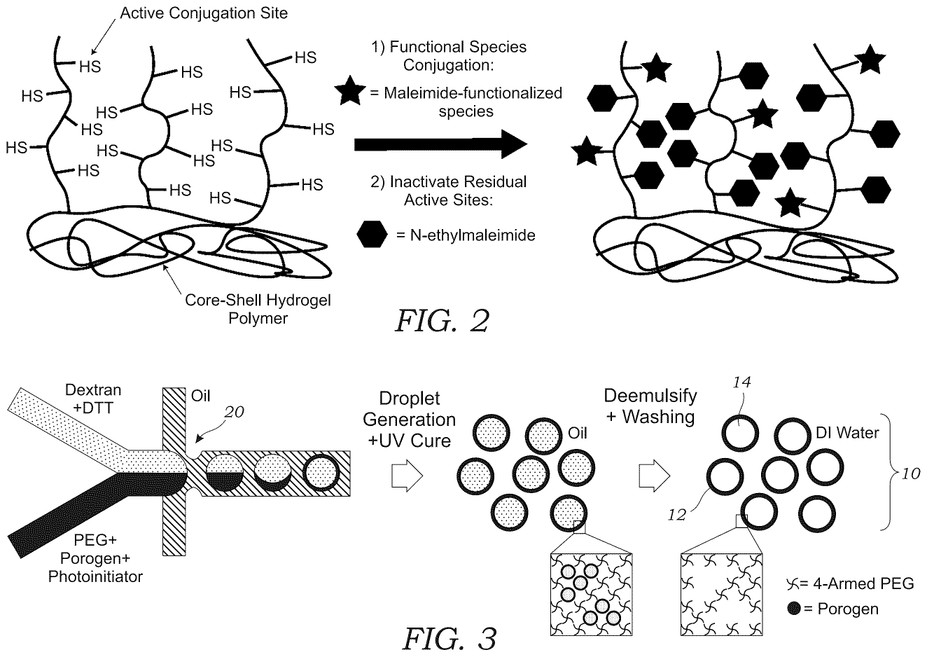

The hydrogel shell 12 can be modified and labelled with fluorescent or colored dyes to aid in analysis. FIG. 2 schematically illustrates how the particles may be modified using free thiol groups. The hydrogel shell 12 can also be tagged with a variety of molecular recognition elements, such as DNA primers to aid in the capture of DNA targets. In some embodiments the core region is pre-loaded with large reactants that cannot transport through the shell 12, such as oligonucleotides, proteins, or enzymes. A plurality of core-shell particles 10 are used as part of a core-shell particle system to perform partitioned reactions. Once mixed with reaction buffer, the plurality of core-shell particles 10 may be from 1% to 99% of the volume of the buffer. The plurality of core-shell particles 10 for reaction preferably includes 10,000 to 500,000 particles 10 per 100 μL of reaction buffer.

### Particle Porosity.

Effective partitioning by the core-shell particles 10 first requires the nucleic acid target 100 of interest to transport into the core region 14 of the particles 10 where it can then be amplified. One can aid the transport of the target nucleic acid 100 by introducing mesoscopic pores into the shell 12 of the particle. Pore size can be modulated by adding a porogen during fabrication process which is illustrated in FIG. 3. In one embodiment, Pluronic F127 was used as a porogen with PEG-norbornene as the polymer precursor due to the similar molecular weight and hydrophilicity as the crosslinked PEG-norbornene. Pluronic F127 is a nonionic triblock copolymer composed of a hydrophobic chain of polyoxypropylene (PPG) with two hydrophilic chains of polyoxyethylene (PEG) attached on either side. Pluronic F127 does not take part in crosslinking but rather can take the spot of the PEG-norbornenes leaving a hole once the hydrogel in crosslinked.

The Flory-Huggins model of crosslinked hydrogels indicates that pore size of a hydrogel is primarily controlled through the chain size of the polymer being crosslinked. This requires a different molecular weight PEG-norbornene for each pore size. By using a porogen instead, punctated voids can be inserted throughout the hydrogel. As porogen concentration increases so do the concentration of the voids. As more voids are created, they become adjacently connected resulting in larger pore sizes. The pore size can be controlled by changing the relative concentration of porogen to PEG-norbornene. This method of porogenation allows for control over pore size without the need to change polymer precursor reagents, only their relative concentrations.

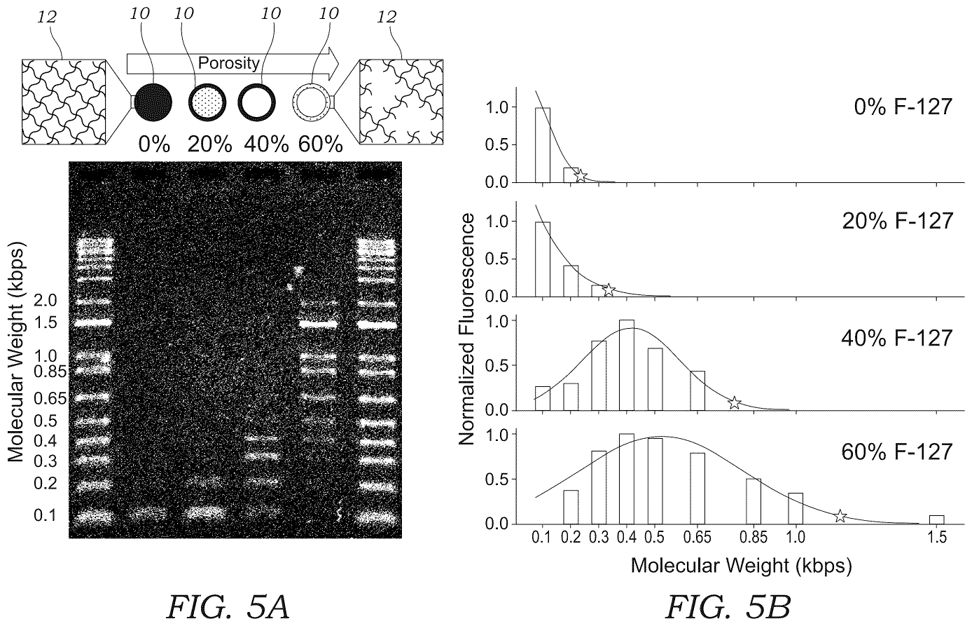

A DNA ladder and gel electrophoresis were used to characterize the porosity of core-shell particles with varying relative porogen concentrations as illustrated in FIG. 5A As the porogen concentration increases, larger DNA bands of the ladder are allowed to diffuse across the shell 12 into the core region 14, indicating larger pore sizes Through quantification of the gel electrophoresis images, it was shown that F127 can be used to modulate pore size and molecular weight cut-off (MWCO) of the particles across almost an entire order of magnitude, from 200 bps to 1200 bps as seen in FIG. 5B. This range of MWCO allows the core-shell particles 10 to be used in a range of clinically relevant targets. Examples include: cfDNA (100-400 bps), Influenza Genome Subunits (2000 bps), genomic DNA (gDNA) cut with restriction enzymes, environmental DNA (eDNA), complementary DNA (cDNA) after reverse transcription, and fragmented or sheared DNA.

### Manufacturing Core-Shell Particles.

Core-shell particles 10 are generated using microfluidic droplet generator devices 20. Microfluidic droplet generator devices 20 may be manufactured using for example polydimethylsiloxane (PDMS) molding techniques. The PDMS devices are cast from a wafer mold using Sylgard 184 in a 1:10 ratio of crosslinker to bulk elastomer. The wafer molds are fabricated using silicon wafers and KMPR 1000 series negative photoresist. Photoresist was applied twice and cured twice using PWM32 Headway Spin Coater and a Karl Suss MA6 aligner, respectively. This results in a mold with two heights. The height at the flow focusing droplet generator 20 was 85 μm and 140 μm at the UV curing channel. The device mask was designed with a flow focusing channel width of 40 μm.

After the PDMS is cast onto the molds, the molds are baked at 65 °C for 4 hours. The individual PDMS devices cut to size. The inlets and outlets are hole punched to accept the precursor tubing. The PDMS devices are then bonded to glass slides. 1×3 in glass slides are placed with the PDMS devices in a PDC-001-HP Harrick Plasma Cleaner, pumped down to 500 mTorr, and plasma cleaned for 1 m and 30 s. The PDMS device is then placed on top of the glass slide, forming a covalent bond. Bonded devices are the baked at 65 °C for 1 hour to strengthen bond.

The bonded devices are then surface treated to increase the hydrophobicity of the microfluidic channel. A 2% (w/w) solution of the Trichloro(1H,1H,2H,2H-perfluorooctyl) silane (Aldrich) in HFE-7500 (3M) was used to flood all the channels of the device. The solution was allowed to react with the surface for 5 minutes before being aspirated out. The channels were then washed with pure HFE-7500 three more times. The devices are then baked at 65 °C for 1 hour to evaporate residual HFE-7500.

Three stock solutions are used for the core-shell particle 10 fabrication, as shown in FIG. 3 (one oil phase and two aqueous-based solutions). The two aqueous based solutions are called precursors. The precursors contain the components needed for crosslinking the particles and polymers required for the aqueous two-phase system that creates the core-shell morphology. Two separate precursor solutions are used in order to keep the components from polymerizing before droplet formation. For one particular formulation the final component concentrations in the droplet prior to crosslinking are: 7.875 wt % 4-Arm PEG Norbornene 10 kDa (Creative PEG Works), 5.25 wt % Pluronic F127 (porogen), 1.5 wt % Lithium phenyl-2,4,6-trimethylbenzoylphosphinate (LAP), 5 wt % Dextran 40 kDa, and 0.30% (w/w) 1,4-Dithiothreitol (DTT). The two precursor solutions are referred to as the dextran precursor and PEG precursor.

The PEG precursor is composed of three components: 4-Arm PEG-norbornene 10 kDa, a crosslinkable polymer; LAP, a photoinitiator; and Pluronic F127, a porogen. To make the PEG precursor, a 3% (w/w) solution of LAP was prepared using PBS. Using the LAP solution, a 17.5% (w/w) 4-Arm PEG Norbornene 10 kDa solution is also prepared. Finally, a 17.5% (w/w) solution of Pluronic F127 is prepared using PBS. The 4-Arm PEG solution and the Pluronic F127 solution is then mixed volumetrically, 60% 4-Arm PEG solution and 40% Pluronic F127 solution. The relative concentration of the 4-Arm PEG solution and Pluronic F127 solution can be changed to modulate the porosity of the resulting particles.

The dextran phase is composed of two components: Dextran 40 kDa and DTT. The dextran is a polymer that interacts with the PEG to setup an aqueous two-phase system. It is not crosslinked and is responsible for the hollow core of the particles. The DTT is small and diffuses into the PEG phase and crosslinks the Norbornene groups of the PEG. To make the dextran precursor, a stock of 5% (w/w) Dextran 40 kDa and 0.75% (w/w) DTT is prepared using PBS. If the porosity is modulated by changing the ratio of 4-Arm PEG to Pluronic F127 in the PEG precursor, then DTT concentration should be similarly changed ratiometrically to compensate for the change in free Norbornene groups.

The oil used for the droplet generation is prepared by diluting 5% Pico-Surf (Sphere Fluidics) into HFE-7500 to create a working solution of 0.25% Pico-Surf in HFE-7500. To fabricate the particles 10, the precursors are fed into the PDMS flow focusing device 20 (FIG. 3) to create droplets. The dextran and the PEG then phase separate in the droplets and are UV crosslinked to lock-in the core-shell structure. To do this, the precursors are each loaded into 1 mL BD syringes. The surfactant oil solution in loaded into a 10 mL BD syringe. Luer stubs are then placed on the syringe and connected to the PDMS devices 20 with Tygon tubing (0.020 I.D.×0.060 O.D.×0.020″ wall diameter) (Saint-Gobain). The Tygon tubing is coupled the PDMS device 20 using a short piece of PEEK tubing (1569 Chromatography Tubing, Orange PEEK, 1/32″ OD×0.020″ ID) (Idex).

The dextran precursor, PEG precursor, and oil syringes are placed in separate Harvard PHD2000 pumps. The oil flow rate is set to 20 uL/min. The dextran precursor flow rate is set to 0.5 uL/min. The PEG precursor is set to 1.5 uL/min. With these precursor stock concentrations, the precursor flow rates can be changed as long as they maintain a 1:3 flow rate ratio.

The PDMS device 20 is placed on a Nikon Eclipse Ti inverted microscope while droplet generation stabilizes. After droplet diameter stabilizes and proper core-shell morphology is observed, the droplets are crosslinked to particles by exposure to UV light. This is done using the DAPI channel of the microscope. The light used is a Lumencor's SOLA Light Engine that produces approximately 40 mW/cm². The PDMS device 20 is positioned so that the UV curing channel is over the 10× objective lens. The microscope is then switched over to the DAPI channel and the PDMS device is illuminated with approximate 350 nm light. The crosslinked particles 10 are then collected.

The collected particles 10 are still in an aqueous-in-oil emulsion and need to be broken out. This is done by first removing all the excess fabrication oil. Being less dense, the particles will cream to the top of the emulsion. Oil on the bottom is removed using gel loading tips. A volume of pure HFE-7500 equal to the amount just removed can be added back to the emulsion. The pure HFE-7500 helps remove surfactant that stabilizes the emulsion. The emulsion is washed twice more with HFE-7500.

A 20% (w/w) solution of 1H,1H,2H,2H-Perfluorooctanol (PFO) is prepared using HFE-7500. A volume of 20% PFO equal to half the volume emulsion is added to the emulsion. For example, if there is 1 mL of particle emulsion 0.5 mL of 20% PFO will be added to the emulsion. A volume of PBS equal to the emulsion is then added to the emulsion. For example, if there is 1 mL of particle emulsion 1 mL of PBS will be added to the emulsion. The emulsion is then slightly agitated causing the particle emulsion to coalesce. Additional PFO solution can be added to increase the rate of coalescence.

Once the emulsion has coalesced and there is no distinct emulsion observed, the Pluronic F127 and dextran can be washed out of the particles 10. A volume of PBS equal to 9 times the volume of the previous emulsion volume is added to the aqueous particle mixture. For example, if there was 1 mL of particle emulsion then 9 mL of PBS will be added to the emulsion. The aqueous particle mixture is then agitated. The particles are spun down and pelleted at 2000 g for 3 minutes. The supernatant is then removed. This washing is repeated until foam is no longer persistent on the aqueous particle mixture.

To remove any remaining HFE-7500 oil droplets, hexane is then added to the aqueous particle mixture. A volume of hexane equal to 2 times the volume of the previous emulsion volume is added to the aqueous particle mixture. For example, if there was 1 mL of particle emulsion then 2 mL of hexane will be added to the emulsion. The mixture is the spun down at 2000 g for 3 minutes. The hexane is aspirated off. The particles are then washed twice more with hexane.

To remove traces of hexane, the particles are then washed with ethanol. Firstly, the aqueous particle mixture is spun down at 2000 g for 3 minutes. Any supernatant is removed, and the particle pellet volume noted. A volume of ethanol equal to 9 times the volume of the particle pellet volume is added to the particle pellet. For example, if there was a 1 mL particle pellet then 9 mL of ethanol will be added to the emulsion. The particle mixture is then agitated. The particles are spun down and pelleted at 2000 g for 3 minutes. The supernatant is then removed. The particles are then washed with ethanol twice more.

To remove traces of ethanol, the particles are then washed with PBS with 0.1% Pluronic F127. Firstly, the aqueous particle mixture is spun down at 2000 g for 3 minutes. Any supernatant is removed, and the particle pellet volume noted. A volume of 0.1% Pluronic PBS equal to 9 times the volume of the particle pellet volume is added to the particle pellet. For example, if there was a 1 mL particle pellet then 9 mL of 0.1% Pluronic PBS will be added to the emulsion. The particle mixture is then agitated. The particles are spun down and pelleted at 2000 g for 3 minutes. The supernatant is then removed. The particles are then washed with 0.1% Pluronic PBS twice more. The particle pellet can be stored at 4 °C for later use.

### Optional Functionalization of Core-Shell Particles.

Due to incomplete crosslinking of all DTT thiol groups, the particles 10 have free thiol groups that can be used as anchorages to conjugate other molecules to, as shown in FIG. 2. These sites are used to conjugate fluorescent dyes or light emitting reporters to the particles 10. To dye the particles 10 a stock solution of 1 mg/mL Alexa Fluor 647 Maleimide (Thermofisher) solution is prepared, Particle pellet is resuspended in PBS with 0.1% Pluronic F127 in a volumetric ratio of 1:3 pellet to PBS. 4 μL of the stock dye solution is added to the particle suspension for every 1 ml, of pelleted particles. Oligonucleotide capture probes, optionally containing a fluorophore, that are functionalized with maleimide can be covalently linked to the particle 10 using a similar protocol. The particle suspension is agitated at 25 °C for 10 min. The rest of the thiols are then capped to prevent interaction with later reagents. The is done by preparing a 100 mM solution of N-ethylmaleimide (Signa). 100 μL of the N-ethylmaleimide is added to the particle suspension for every 1 mL of initial particle pellet. The N-ethylmaleimide is allowed to react with the particles for 2 hours at 25 °C. The particles are then pelleted at 2000 g for 3 minutes. The supernatant is removed and replaced with PBS with 0.1% Pluronic F127. The particles are washed twice more in this manner. The supernatant is removed, and particle pellet is stored at 4 °C for later use.

### Example Digital Nucleic Acid Amplification Test

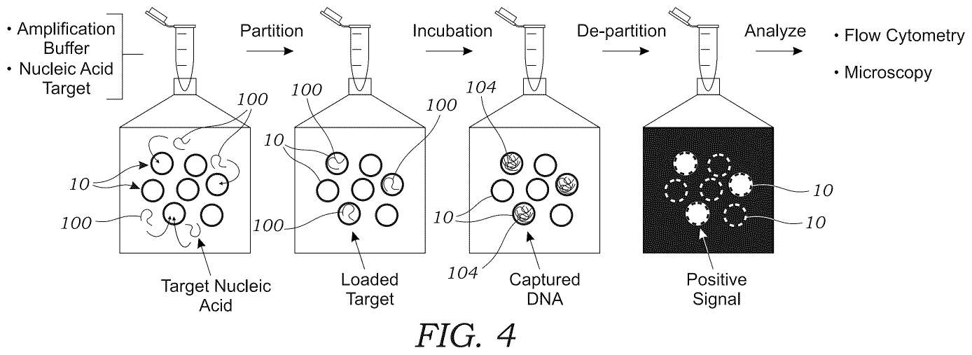

To perform loop-mediated isothermal amplification (LAMP) using the particles 10 using the workflow illustrated in FIG. 4, a LAMP reaction buffer is prepared. This buffer is composed of 100 mM Tris-HCl, 50 mM KCl, 50 mM (NH4)₂SO₄, 40 mM MgSO₄, 5M Betaine, 0.005% (v/v) Triton-X 100. Before the LAMP reaction buffer is diluted to its finally concentration the pH is adjusted to 8.8 with 1M NaOH.

To perform loop-mediated isothermal amplification, a primer stock solution is also needed. The LAMP primer set is composed of six ssDNA fragments: forward inner primer (FIP), backward inner primer (BIP), forward loop primer (F Loop), backward loop primer (B Loop), forward outer primer (F3), backward outer primer (B3). The primers were synthesized by Thermofisher. Each primer was rehydrated to a concentration of 500 uM and then combined in a final stock solution containing: 48 uM FIP, 48 uM BIP, 12 uM F Loop, 12 uM B Loop, 6 uM F3, and 6 uM B3. The sequence of each primer is as shown for the BRCA1 target DNA sequence 100:

FIP:
[SEQ ID NO: 1]
GACAGGCTGTGGGGTTTCTCTCCCGGGACTCTACTACCTTT
BIP:
[SEQ ID NO: 2]
GTAATTCCCGCGCTTTTCCGTCTGTCCCTCCCATCCTCTG
F Loop:
[SEQ ID NO: 3]
GAAATCCACTCTCCCACGCC
B Loop:
[SEQ ID NO: 4]
CAATCCAGAGCCCCGAGAGA
F3:
[SEQ ID NO: 5]
TCAGGAGGCCTTCACCCTC
B3:
[SEQ ID NO: 6]
GGAAACCAAGGGGCTACCG

The DNA target 100 used to test LAMP amplification is a segment of the Breast Cancer gene 1 (BRCA1). The target nucleic acid sequence 100 was synthesized by Thermofisher. The target 100 was rehydrated to a concentration of 4.9e9 cps/uL. The sequence is [SEQ ID NO:7]

To perform an assay using the core-shell particles 10, LAMP reaction buffer, primer stock, dNTP stock, Bts 2.0 polymerase (New England Biolabs), bovine serum albumin (BSA) (New England Biolabs), and DNA sample are combined in the following volumetric combination: 20% LAMP reaction buffer, 8% dNTP stock, 3.3% primer stock, 1.3% Bts 2.0 polymerase, 5% BSA, 52.3% particles, and 10% DNA sample. This results in approximately 50,000 particles 10 per 100 μL of completed LAMP reaction mix.

The complete sample solution is prepared by mixing the particles 100 with the DNA sample 102. This is allowed to incubate at 25 °C for 10 mins to enable DNA to equilibrate and transport into core-shell particles 10. The LAMP reaction buffer is then added to the sample solution followed by the dNTP stock, primer stock, polymerase, and BSA.

The mixture is then spun down at 2000 g for 3 minutes. The supernatant then is removed leaving behind the particles pellet. Bio-Rad's Droplet Generation Oil for Probes is added to the pellet in a 2:1 ratio of oil to pellet. The mixture is then pipetted to emulsify the particles 10, creating an aqueous-in-oil emulsion. The particles 10 act as uniform templates that partition droplets in which the LAMP amplification can take place. The emulsion is then incubated in an Eppendorf Mastercycler Personal at 65 °C with a lid temperature of 67 °C for 3 hours.

The collected particles 100 with amplified DNA or amplicons 104 are still in an aqueous-in-oil emulsion and are broken out for downstream analysis by flow cytometry and imaging. This is done by first removing all the excess oil. Being less dense, the particles 100 will cream to the top of the emulsion. Oil on the bottom is removed using gel loading tips. A volume of pure HFE-7500 equal to the amount of oil just removed can be added back to the emulsion. The pure HFE-7500 helps remove surfactant that stabilizes the emulsion. The emulsion is washed twice more with HFE-7500.

A 20% (w/w) solution of 1H,1H,2H,2H-Perfluorooctanol (PFO) is prepared using HFE-7500. A volume of 20% PFO equal to half the volume emulsion is added to the emulsion. For example, if there is 1 mL of particle emulsion 0.5 mL of 20% PFO will be added to the emulsion. A volume of PBS equal to the emulsion is then added to the emulsion. For example, if there is 1 mL of particle emulsion 1 mL of PBS will be added to the emulsion. The emulsion is then slightly agitated causing the particle emulsion to coalesce. Additional PFO solution can be added to increase the rate of coalescence.

To remove any remaining HFE-7500 oil droplets, hexane is then added to the aqueous particle mixture. A volume of hexane equal to 2 times the volume of the previous emulsion volume is added to the aqueous particle mixture. For example, if there was 1 mL of particle emulsion then 2 mL of PBS will be added to the emulsion. The mixture is the spun down at 2000 g for 3 minutes. The hexane is aspirated off. The particles are then washed twice more with hexane.

To remove traces of hexane, the particles are then washed with ethanol. Firstly, the aqueous particle mixture is spun down at 2000 g for 3 minutes. Any supernatant is removed, and the particle pellet volume noted. A volume of ethanol equal to 9 times the volume of the particle pellet volume is added to the particle pellet. For example, if there was a 1 mL particle pellet then 9 mL of ethanol will be added to the emulsion. The particle mixture is then agitated. The particles are spun down and pelleted at 2000 g for 3 minutes. The supernatant is then removed. The particles are then washed with ethanol twice more.

To remove traces of ethanol, the particles are then washed with PBS with 0.1% Pluronic F127. Firstly, the aqueous particle mixture is spun down at 2000 g for 3 minutes. Any supernatant is removed, and the particle pellet volume noted. A volume of 0.1% Pluronic PBS equal to 9 times the volume of the particle pellet volume is added to the particle pellet. For example, if there was a 1 mL particle pellet then 9 mL of 0.1% Pluronic PBS will be added to the emulsion. The particle mixture is then agitated. The particles are spun down and pelleted at 2000 g for 3 minutes. The supernatant is then removed. The particles 10 are then washed with ethanol twice more. The particle pellet can be stored at 4 °C for later use.

Washing steps can be performed in an automated fashion using for example automated instrumentations for centrifugation and washing (e.g., Beckman Coulter LyseWash Assistant). Optionally, the particles 10 may be subject to a desiccation process to remove water from the particles 10. Desiccation increases the shelf life of the particles 10 and minimizes the amount of needed reagents for the end user. To desiccate the particles 10, the particles 10 are initially contained in an aqueous PBS solution following fabrication. These particles 10 undergo three washing steps to prepare them for desiccation. During each washing step, the particles 10 are pelleted and the supernatant is discarded. A desiccation solution is then introduced, comprised of acetone with 20% by weight of PEG-800. This solution plays a dual role; the acetone effectively extracts the water content from the particles 10, while the PEG-800 ensures morphological integrity by preventing the complete collapse of the desiccated particle 10. As a result of this process, the particles 10 experience a reduction in size. Specifically, before desiccation, the mean diameter of the particles 10 is 44.03 μm with a standard deviation of 1.76 (n=50), while post-desiccation, the mean diameter of the particles 10 reduces to 24.40 μm with a standard deviation of 1.94 (n=50). This desiccation not only aids in shelf-life enhancement but also ensures that when integrated into assays, the particles 10 actively absorb the necessary components without introducing undue volume, thus eliminating the need for assay adjustments and reducing supernatant wastage

### Core-Shell Particle Analysis.

The particles 10 can then be analyzed using complementary oligonucleotide strands with fluorescent dyes or an intercalation dye. Staining with an intercalation dye is done by preparing an intercalation dye solution. This solution is composed of 0.1% Pluronic F127 in PBS with SYBR Green 1 (Sigma) diluted to a 1× concentrations. The particles 10 are spun down and pelleted at 2000 g for 3 minutes. The supernatant is then removed and replaced with the intercalation dye solution. The nucleic acids associated with the particles 10 is now stained and can be analyzed.

Particles can be optically interrogated (e.g., imaged) and analyzed with fluorescence microscopes. For microscope analysis, the particles 100 can be place onto a glass slide, or well plate. If well plates are used, allow particles to settle for 3 minutes prior to imaging. Images are taken using a 10× objective lens. Images are taken through FITC and TRITC filter sets using a camera. The dyed particles 10 fluoresce in the TRITC channel while the stained nucleic acids or amplicons 104 fluoresce in the FITC channel. The images are analyzed by a program that counts the total number of particles 10 in the TRITC channel. Each particle in the TRITC channel is then used to make a mask to extract the nucleic acid fluorescence intensity from the FITC channel.

Particle analysis can be performed using flow cytometers and fluorescence activated cell sorters. For analysis on the Sony SH800 flow cytometer, the concentration of the particles is adjusted to 100,000 particles per milliliter. All lasers were turned on and signal was captured on all filters. The events were triggered off the forward scatter. Particles 10 were analyzed and identified by using the area of the FL3 channel and the presence of nucleic acids was quantified using the area of the FL2 channel.

For analysis on the On-Chip flow cytometer, the concentration of the particles 10 is adjusted to 50,000 particles per milliliter. All lasers were turned on and signal was captured on all filters. The events were triggered off the forward scatter. Particles were analyzed using the height of the FL2 channel and the presence of nucleic acids was quantified using the height of the FL4 channel.

To quantify nucleic acid concentration within the particles 10, Poisson partitioning statistics are utilized. The data is derived from fluorescence intensity, obtained either via microscopy or flow cytometry. Particles 10 displaying fluorescence intensity above a specific threshold are identified as positive, indicating the presence of amplified entrapped nucleic acids. This threshold is established by analyzing a sample of core-shell particles 10 devoid of nucleic acids, subsequently pinpointing a fluorescence level that excludes 99.9% (or another preset threshold) of negative instances from this sample 102.

For determining the size of the core region 14 within the particle 10, two distinct methodologies are employed:

Direct Measurement: Post-fabrication imaging is conducted to precisely gauge the core size. See paragraph under heading Measurement of Core Volume of Core-Shell Particles for details.

Indirect Measurement: Here, images captured during the fabrication process, specifically prior to the particle crosslinking, are employed to estimate core size. While potentially less accurate than the direct approach, it omits the need for an additional imaging step.

Applying the established fluorescence threshold to the nucleic acid-containing sample 102, data is categorized into two groups: an ‘on’ population, with particles 10 surpassing the threshold, and an ‘off’ population, with particles 10 falling below the threshold. The negative natural logarithm of the ratio between the ‘off’ population and the total particle population yields the count of nucleic acid targets in sample 102 that underwent amplification within each core-shell particle 10. This value, when divided by the determined volume of the core region 14 (either by direct or indirect means), provides the nucleic acid target concentration. This computation can be auto-executed via the microscope, optical imaging device, or a separate computational device.

$$Targets per Partition = - ln ( \frac{‘ off ’ Population}{Total Population} ) \qquad ( 1 )$$

$$Nucleic Acid Concentration = \frac{Targets per Partition}{Core Volume} \qquad ( 2 )$$

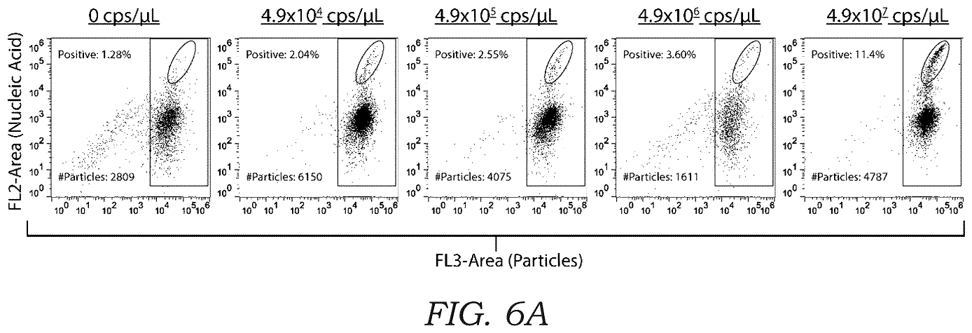

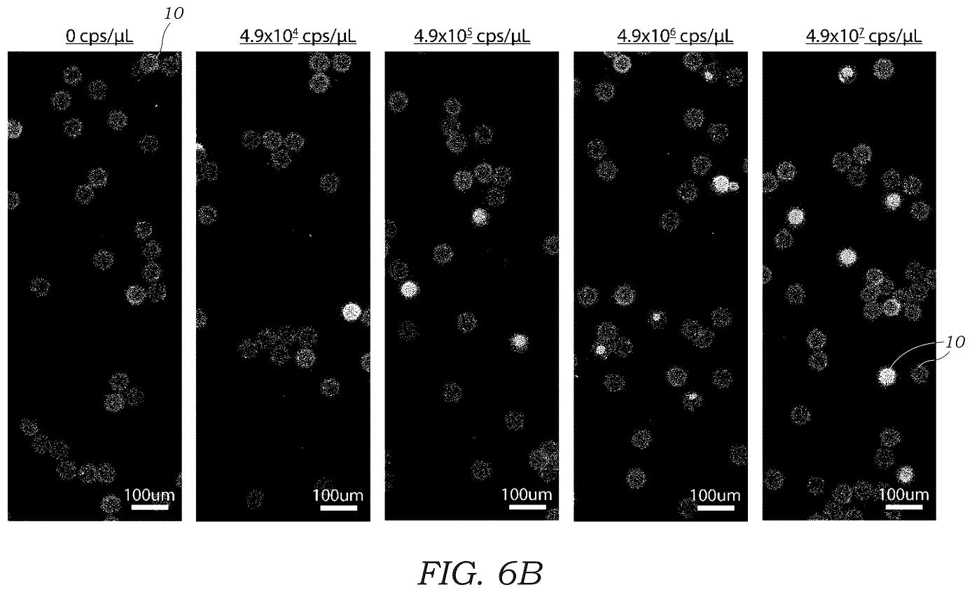

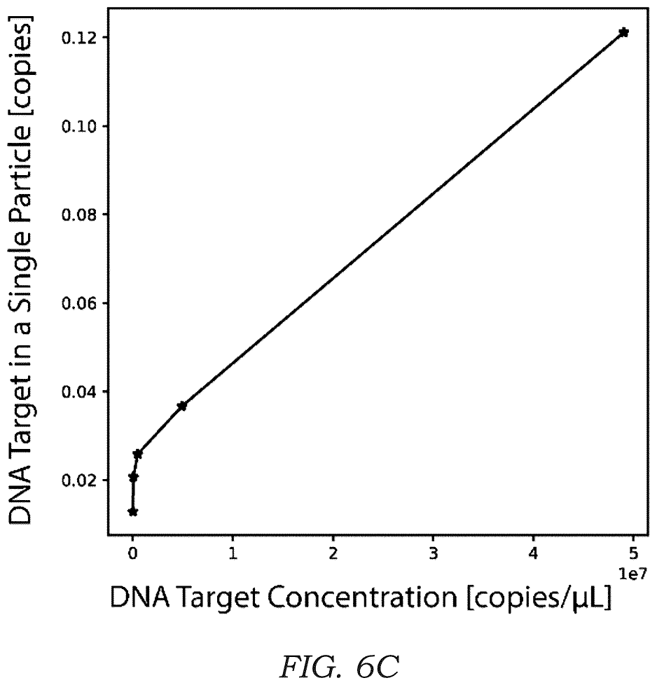

To measure sensitivity on the Sony SH800 varying concentrations of the BRCA1 target 100 were incubated with core-shell particles 10 and a digital LAMP assay was performed as described herein. The target concentrations used in final particle mix were: 0, 4.9e4, 4.9e5, 4.9e6, 4.9e7 copies/μL. The resultant particles 10 were analyzed as indicated above. The gating used to separate the total particle and positive particle populations can be seen in FIG. 6A. Sample microscopic images of each condition were also taken and shown in FIG. 6B. FIG. 6C show each condition plotted with the prepared DNA target concentration compared against its measured target per partition concentration. The limit-of-detection was measured to be 0.012 targets per partition.

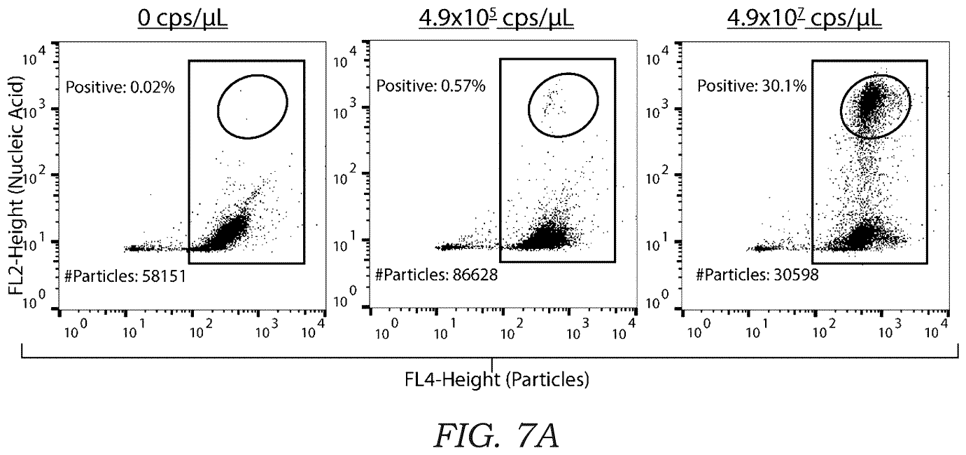

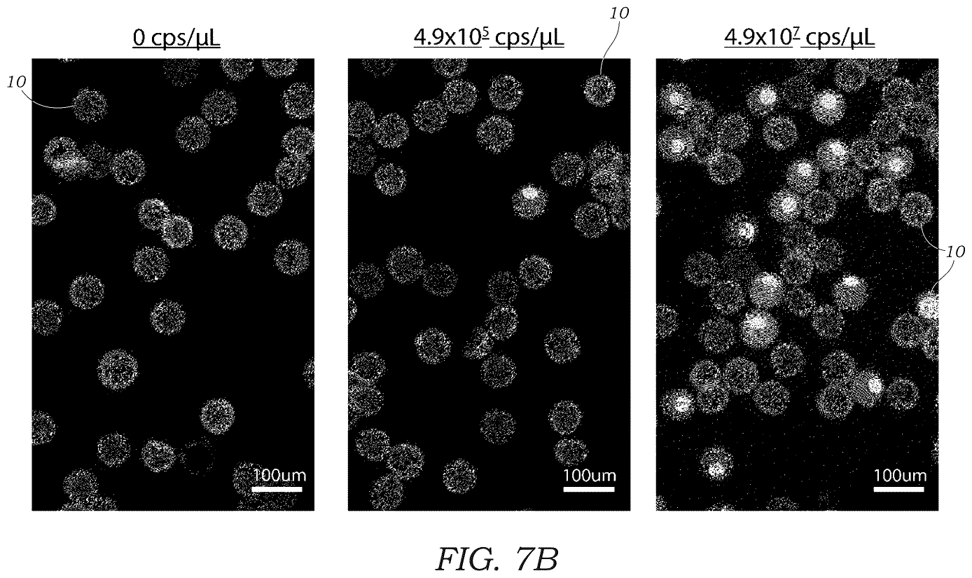

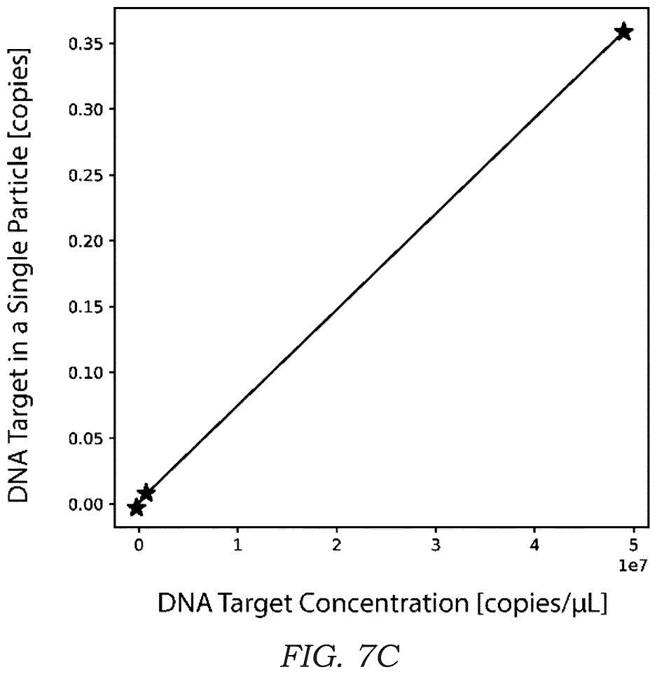

To measure sensitivity on the On-Chip a sweep across varying concentrations of the BRCA1 target were performed. The target concentrations used in final particle mix were: 0, 4.9e5, 4.9e7 copies/uL. The resultant particles were analyzed as indicated above. The gating used to separate the total particle and positive particle populations can be seen in FIG. 7A. Sample microscopic images of each condition were also taken and shown in FIG. 7B. FIG. 7C show each condition plotted with the prepared DNA target concentration compared against its measured target per partition concentration. The limit-of-detect was measured to be 2.29e-4 targets per partition.

### Particle Porosity Measurements.

The porosity of core-shell particles 10 with different amounts of porogen was measured by mixing 100 ul of particles 10, 1 uL 1 kb Ladder Plus, and 100 uL PBS. The mixture was allowed to sit for 10 mins. The particles 10 were then washed 3× times with DI water. The particles 10 were then spun down and the supernatant removed. 100 μL of 3×PBS added to the pellet and vortexed to elute the DNA. The particles 10 were allowed to incubate at 25 °C for 10 min. The particles 10 were spun down and gel electrophoresis was run on the supernatant.

Electrophoresis gels were made by dissolving 7 g agarose (Sigma) in 70 mL warm of TBE buffer (Sigma) and adding 7 uL of SYBR Green I. Samples were loaded using Gel Loading Buffer (Sigma). Electrophoresis was run at 95V for 45 min using a PowerPac Universal power supply (Rio Rad). Photos of the gels were analyzed using a Python script.

Particle were fabricated using four different Pluronic F127 porogen to PEG-Norbornene ratios: 0%, 20%, 40%, and 60%. Their pore size was characterized using the DNA ladder technique described above. A DNA ladder and gel electrophoresis were used to characterize the porosity of core-shell particles with varying relative porogen concentrations as seen in FIG. 5A. As the porogen concentration increases, larger DNA bands of the ladder are allowed to diffuse across the shell 12 into the core region 14, indicating larger pore sizes. Through quantification of the gel electrophoresis images, it was shown that F127 can be used to modulate pore size and molecular weight cut-off (MWCO) of the particles 10 across almost an entire order of magnitude, from 200 bps to 1200 bps corresponding to 120 kD to approximately 600 kD, The MWCO was calculated by fitting a curve to the gel electrophoresis image data and find the largest molecular weight that is 10% of the maximum value of the fitted curve, indicated by the diamond in FIG. 5B.

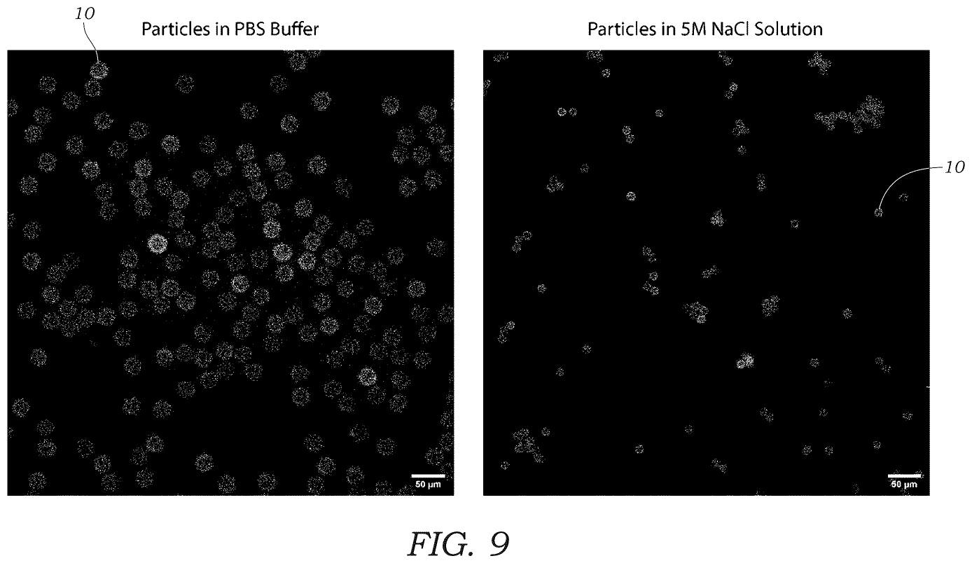

The size of the particles may also be modulated after running the assay by changing the ionic strength of the solution the particles are stored in. FIG. 9 illustrates that 60 μm core-shell particles 10 in PBS buffer can be shrunk down to ~30 μm once placed in a 5M NaCl solution. By shrinking the particles 10 in size, the contents sequestered in their cores 14 are concentrated. This can increase the local amplified intensity of fluorescent intercalator dyes or other analyte detection dyes, making the signal measurable on less sensitive detectors. By using ionic strength to modulate the size of the particles 10, the particle 10 can be made to fit into detectors and flow cytometers that would have fluidic channels or conduits otherwise be too small for the particles 10 to pass through.

Alternate amplification methods can be used to produce amplified signal within core-shell particles 10. In one embodiment, rolling circle amplification (RCA) may be used. RCA is similar to LAMP in that it also produces amplification products or amplicons 104 that are larger than the original nucleic acid target 100. These amplification products can contain 10s to 100s of concatenated repeats of the template target molecule. This allow these products to also be trapped in the core 14 of the particle 10 for analysis.

In another embodiment, PCR may be used. The amplification products of PCR are the same size or shorter than the original nucleic acid target 100. Therefore, primers used for PCR or other complementary oligonucleotide capture molecules can be conjugated to the core-shell particles 10 within the core region 14 to capture the resultant amplification products for downstream labeling and detection. Alternatively, the primers or other target binding moiety can be localized to the core 14 of the core-shell particle 10 and conjugated to large molecules above the molecular weight cut off to prevent transport across the shell 12. These large molecules may include high molecular weight sugars like dextran, large proteins, nanoparticles, or the like.

To attach primers to the hydrogel shell 12 of the particles 10, biotinylated or thiol-terminated oligonucleotides can be used. For biotinylated primer attachment, a 100 mM stock of biotin-maleimide is first made. Prior to N-ethylmaleimide capping, the 100 uL of the stock biotin-maleimide is added to every 1 mL of particles 100 and incubated at room temperature for 2 hours. A volume of 0.1% Pluronic PBS equal to 9 times the volume of the particle pellet volume is added to the particle pellet. For example, if there was a 1 mL particle pellet then 9 mL of 0.1% Pluronic PBS will be added. The particle mixture is then agitated. The particles 10 are spun down and pelleted at 2000 g for 3 minutes. The supernatant is then removed. The particles are then washed with 0.1% Pluronic PBS twice more. 100 uL of a 1 mg/mL streptavidin solution can is then added the particle for every 1 mL of pelleted particles. The particles are then incubated at room temperature for 1 hour while slowly agitating them. The particles are then washed three times in a similar manner as the last washing step. The streptavidin-coated particles can be stored at 4 °C for later biotinylated primer or other target binding moiety conjugation.

To run PCR using the streptavidin-coated core-shell particles 10, the follow components are assembled to create a PCR master mix: 50 uL of ReadyMix Taq PCR Reagent Mix (Sigma), 1 uL of 1.0 uM forward primer, 1 uL of 1.0 uM reverse primer, 8 uL of BRCA sample, 40 uL of streptavidin-coated particles. The PCR master mix is allowed to incubate at room temperature for 10 minutes. All or a fraction of the oligonucleotides with the forward primer sequence are tagged with a biotin that will get incorporated in the particle hydrogel shell 12 during amplification. The PCR primers for one example target are as shown (Thermofisher):

Forward Primer:
[SEQ ID NO: 8]
Biotin-TCAGGAGGCCTTCACCCTC
Reverse Primer:
[SEQ ID NO: 9]
GGAAACCAAGGGGCTACCG

In some embodiments, the primers or target binding moieties are associated with the core-shell particles 10 prior to mixing with PCR reaction mix. Different subsets of core-shell particles 12 may also be labeled with different primers that amplify different targets and may be labeled with different dyes that act as an optical barcode for the presence of a specific primer.

For PCR amplification the PCR mixture is then spun down at 2000 g for 3 minutes. The supernatant then is removed leaving behind the particle pellet. Bio-Rad Droplet Generation Oil for Probes is added to the pellet in a 2:1 ratio of oil to pellet. The mixture is then pipetted to emulsify the particles 10, creating an aqueous-in-oil emulsion. The particles 10 act as uniform templates that partition droplets in which the PCR amplification can take place. The emulsion is thermally cycled in an Eppendorf Mastercycler Personal for 35 cycles with the temperature parameters shown:
Denature: 94 °C for 1 minute
Annealing: 55 °C for 2 minutes
Extension: 72 °C for 3 minutes.

Once DNA is amplified in the core-shell particles 10, the core-shell particles 10 are processed in a similar manner as described for the LAMP procedure described herein.

### ATPS-Based Production of Core-Shell Droplets.

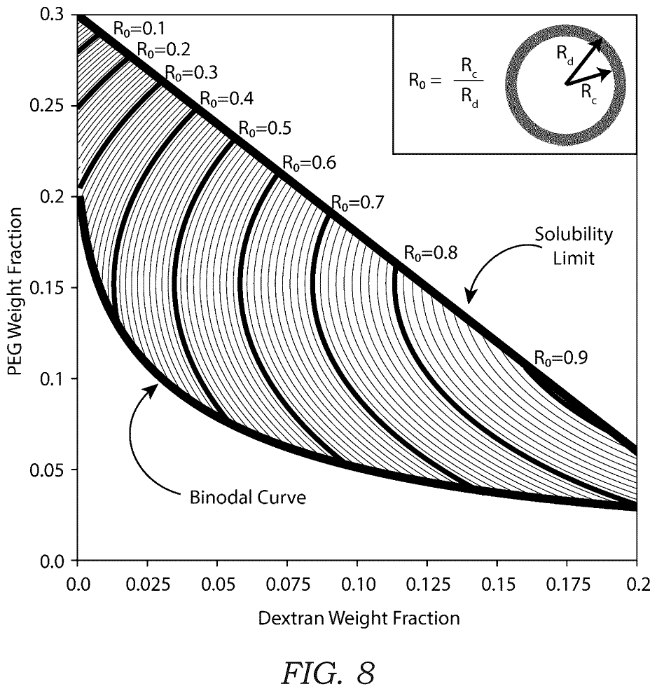

As described, the core-shell morphology is achieve using an aqueous two-phase system (ATPS) during fabrication. ATPSs are created by combining two or more water soluble compounds in solution. For example, PEG and Dextran, PEG and gelatin, or PEG and NaCl. As an example, PEG and Dextran are combined inside a droplet. FIG. 8 illustrates a binodal curve indicating an interface in which an ATPS composed of 4-arm 10 kDa PEG-Norbornene and 40 kDa Dextran will phase separate. Due to molecular interactions the solutions separate into two separate phases. The PEG layer phase separates to the outside of the droplet near the water-oil interface, while the dextran stays at the core. The functionalized PEG is then crosslinked to form a hydrogel shell 12. The droplet is then broken out of emulsion and the dextran is washed out of the center of the particle, producing a core-shell particle 10.

When fabricating core-shell particles 10 using an aqueous two-phase process, the porosity of the shell 12 of the particles 10 can be modified to allow a range of smaller molecular weight molecules to transport in and out of the shell particle 10 while restricting or trapping larger molecular weight molecules. In some embodiments, a porogen can be added to the precursor cross-linkable polymer solution before generation of droplets, phase separation, and crosslinking. These porogens should be configured to phase separate into the cross-linkable phase. For example, the porogens should have similar chemical moieties to the cross-linkable phase. For example, for a cross-linkable polymer solution comprising polyethylene glycol (PEG), the porogen may also comprise PEG moieties. These porogens do not partake in crosslinking and therefore leave a void behind. The concentration of the porogen can be increased to increase the number of voids in the final shell 12. As the number of voids increase the so do their connection with adjacent voids which leads to larger pore size and a larger molecular weight threshold for transport through the shell 12. This creates larger pore sizes.

In one embodiment, the cross-linkable polymer solution comprises different molecular weight precursor materials to tune the porosity of the polymer shell 12. In a non-limiting, example, a particle's shell 12 comprises 10 kDa 4-arm PEG-norbornene. By increasing the size of the crosslinked polymer, the distance between adjacent polymer molecules increased. This is in agreement with the Flory-Huggins model of crosslinked hydrogels. It indicates that pore size of a hydrogel is primarily controlled through the chain size of the polymer being crosslinked. The increase in gaps between polymer molecules, increases the pore size of the resultant hydrogel.

The core-shell particles 10 can be fabricated by many different methods. In one embodiment, flow-focusing droplet generation may be used to make spherical droplets in which the hydrogel is cross-linked. The precursor reagents required for crosslinking and obtaining a core-shell particle morphology can be coflowed together before the flow-focusing droplet generator 20. After droplet generation the droplets can be crosslinked into particles by UV light, pH modulation, temperature modulation, or other polymerization initiation methods well known in the art.

In another embodiment, step-emulsifier droplet generation may be used to make spherical droplets in which to cross-link the hydrogel. The precursor reagents required for crosslinking and obtaining a core-shell particle morphology can be mixed in a single phase before being fed into the step-emulsifier. After droplet generation and phase separation the droplets can be crosslinked into particles by UV light, pH modulation, temperature modulation, or other polymerization initiation methods well known in the art.

Controlling the particle morphology is required for accurate encapsulation of reaction products to perform downstream analysis in the case where analysis is performed after breaking the emulsion. In some embodiments, outside diameter can be determined by the precursor droplet diameter prior to crosslinking. Droplet diameter can be controlled by the droplet generator 20 dimensions. Droplet dimension can also be controlled by varying the oil and precursor concentrations during droplet generation.

Morphology, such as the shell thickness and cavity size, can be controlled through the composition of the aqueous two-phase system. In some embodiments, precursor composition can be modified to control the relative phase volume of the resultant aqueous two-phase system. By controlling relative phase volumes, the shell thickness can be selected. In a similar manner the inner core volume can also be selected by controlling relative phase volumes. Shell thicknesses of at least >2 micrometers are preferred to maintain particle mechanical stability. In some embodiments, shell thicknesses of >5 micrometers are preferred or even >10 micrometers are preferred to reduce the transport of reaction products out of the particles 10, when oils are not used to form an emulsion and partition separate hollow particles. In these embodiments, longer incubation times of >10 minutes may be used to ensure target molecules 100 have sufficient time to transport through larger shell thicknesses into the internal volume of the core-shell particle 10.

### Measurement of Core Volume of Core-Shell Particles

The volume of the core region 14 used in equation (2) above may be determined by direct observation of the final core-shell particles 10 that are generated. For example, for a particular batch of core-shell particles 10 a representative group of core-shell particles 10 may be observed under a microscope where the volume may be directly ascertained by measuring the radius or diameter of the core region 14. The volume may then be directly calculated (i.e., V=4/3πr³). Typically, there is minimal variability within a single batch, although one may use an average or median volume which can then be applied to the particular batch of core-shell particles 10. Alternatively, the volume of the core region 14 may be estimated or indirectly calculated based on images of the core-shell particles 10 obtained prior to or during crosslinking. For example, a microscope can be used to both crosslink (via UV light) and image the just formed core-shell particles 10. This approach helps in reducing the need for subsequent imaging post-fabrication. The swelling ratio, which is used to determine core size, is affected by several components including the polymers used, ATPS (Aqueous Two-Phase System/Separation), and the method of crosslinking, such as UV light or changes in pH. ATPS particularly determines the relative lengths of the particle radius and its core radius. By integrating the swelling ratio, ATPS, and images acquired during fabrication, one can empirically determine/calculate the core radius/volume.

### Methods of Partitioning Core-Shell Particles

Creating individual discrete partitions after the target 100 has been loaded into the particles 10 is of import for digital assays. In one embodiment, the particles 10 can be used to template a water-in-oil emulsion. Once the target 100 has been loaded into the particles 10, oil and surfactant can be added before being agitated. This results in uniform droplets containing the core-shell particles 10. The continuous oil phase prevents the targets 100 or products from target-specific reactions from diffusing into adjacent particles 10.

As an alternative to using oils to partition, the characteristics of a bulk aqueous phase in between the particles 10 can also be modified to prevent transport of the target 100 from one particle 10 to an adjacent particle 10. In one embodiment, a compound can be added to the aqueous bulk phase to reduce diffusion rates and reaction kinetics in the bulk phase. In a non-limiting example, Ficoll or a high-molecular weight dextran may be used. Due to the high molecular weight of the added compound, it does not diffuse into the particles 10 and therefore does not inhibit the amplification process in the core 14 of the particles 10.

In another embodiment, a chelator may be added to scavenge any target transiting the bulk aqueous phase outside of the core-shell particles 10. A non-limiting example of this is the use of DEAE Dextran or other positively charge polymer. The positively charged polymer can form complexes with nucleic acids preventing them from diffusing or transporting further. Due to the high molecular weight of the added compound, it does not diffuse into the particles 10 and therefore does not inhibit the amplification process in the core region 14 of the particles 10.

Diffusion of the target 100 between adjacent particles 10 can be mitigated by selecting a short amplification runtime. In this system there are two competing kinetic factors. The rate of amplification of the target 100 and the diffusion or other transport of the target 100 or short amplicons 104 to adjacent particles 100. An amplification cut-off time can be chosen to permit sufficient amplification of the target and creation of amplicons 104 larger than the pore cutoff while preventing a significant portion of the smaller amplification products to diffuse to adjacent particles 10 and create larger enough amplicons 104 to remain entrapped in adjacent particles 20.

In another embodiment, the use of smart sensing hydrogels can be used to prevent the transport of targets 100 or small amplicons 104 in between adjacent particles 10. In one non-limiting example, Poly(N-isopropylacrylamide) (PNIPAM) may be incorporated in the cross-linked hydrogel of the shell 12 of the particle. As the temperature raises (e.g., from incubation) the PNIPAM polymer undergoes a phase change to reduce the pore size as the shell 12 shrinks, preventing the target 100 or small amplicons 104 from diffusing out. In another non-limiting example, polyacrylic acid may be incorporated into the hydrogel of the particle's shell 12. This sensitizes the pore size to the pH of the surrounding aqueous bulk phase. As the target amplifies the pH of the bulk phase can increase due to the byproducts of the amplification process. This causes the polyacrylic acid to shrink the pore size of the shell 12, preventing the diffusion of the target 100 and its amplification products to adjacent particles. In another non-limiting example, charged moieties may be added to the hydrogel of the particle shell 12, such as amine groups or carboxylate groups. The pore sizes of the particle shells 12 are then dependent on the ionic strength of the bulk aqueous phase. As the target amplifies the ionic strength of the bulk phase can increase due to the byproducts of the amplification process. This causes the charged polymer to shrink the pore size of the shell 12, preventing the target 100 and its amplification products 104 from transporting to adjacent particles 10.

The partitioning efficiency (average target concentration inside particle 10 over target concentration of entire reaction) can be increased be modifying the components of the particles 10. In one embodiment, primers or oligonucleotide capture moieties can be attached to the shell 12 of the particle 10 as described herein. By binding target nucleic acids to the hydrogel shell 12, they are pulled out of the surrounding solution increasing the concentration inside the particles 10 and increasing loading efficiency.

In another embodiment, the core-shell particle 10 can be fabricated with a positive charged polymer, like DEAE Dextran, in the core of the particle 10. A high molecule weight can be chosen to prevent the positively charged polymer from diffusing out of the particle 10. The trapped positively charged polymer can then associate with negatively charged nucleic acids out of the surrounding solution. Thereby, increasing the concentration of target nucleic acids 100 inside the particles and increasing loading efficiency.

While embodiments of the present invention have been shown and described, various modifications may be made without departing from the scope of the present invention. In addition, the features of the various embodiments described herein may be used or interchanged with other embodiments. The invention, therefore, should not be limited, except to the following claims, and their equivalents.

1. A core-shell particle system for performing amplification of a target nucleic acid comprising:
a plurality of particles comprising a hydrogel outer shell surrounding a core region that is substantially devoid of hydrogel, wherein the hydrogel outer shell comprises pores having a base pair length cut-off within the range of 200 bps to 1200 bps that permits the passage of the target nucleic acid and nucleic acid amplification reagents into the core region from an external environment surrounding the plurality of particles while substantially preventing the escape of amplicons generated in the core region in response to nucleic acid amplification of the target nucleic acid.

2. The core-shell particle system of claim 1, wherein the plurality of particles have a diameter within the range of 10 μm to 300 μm.

3. The core-shell particle system of claim 1, wherein the outer shells of the plurality of particles have a thickness within the range of 0.5 μm to 100 μm.

4. The core-shell particle system of claim 1, wherein the plurality of particles are conjugated to a light emitting reporter or dye.

5. The core-shell particle system of claim 1, wherein the target nucleic acid comprises one of cfDNA, gDNA, eDNA, cDNA, DNA digested or broken into smaller length units, or viral subunits.

6. The core-shell particle system of claim 1, wherein the plurality of particles are partitioned from one another with an oil phase.

7. The core-shell particle system of claim 1, wherein the plurality of particles are partitioned from one another with an aqueous phase containing a high-molecular weight polysaccharide.

8. The core-shell particle system of claim 1, wherein the plurality of particles are partitioned from one another with an aqueous phase containing a target nucleic acid chelator.

9. The core-shell particle system of claim 1, wherein the plurality of particles comprise poly(N-isopropylacrylamide) (PNIPAM).

10. The core-shell particle system of claim 1, wherein the plurality of particles comprise oligonucleotides conjugated to the outer shells within the core regions.

11. The core-shell particle system of claim 1, wherein the core regions of the plurality of particles comprises a positively charged polymer contained therein.

12. A method of using the core-shell particle system of claim 1 comprising:
mixing the plurality of particles with a sample containing the target nucleic acid and nucleic acid amplification reagents;
partitioning the plurality of particles from one another;
incubating the plurality of particles for a period of time so as to generate target amplicons;
de-partitioning the plurality of particles from one another;
exposing the de-partitioned plurality of particles to a dye or fluorescent reporter that labels nucleic acids or is specific to a target amplicon; and
optically interrogating the plurality of particles.

13. The method of claim 12, wherein the plurality of particles are optically interrogated with a microscope or an imaging device.

14. The method of claim 12, wherein the plurality of particles are optically interrogated with a flow cytometer or a fluorescence activated cell sorter.

15. The method of claim 12, further comprising calculating a concentration of the target nucleic acid based at least in part on the fraction of particles that exhibit a positive or negative signal above or below a threshold in response to optical interrogation.

16. The method of claim 12, wherein the plurality of particles are conjugated with a light emitting reporter that emits light in a different wavelength or wavelength range as compared to the dye or fluorescent reporter.

17. The method of claim 12, wherein prior to optical interrogation, the plurality of particles are exposed to a solution of differing ionic strength to reduce the size of the plurality of particles.

18. The method of claim 12, wherein the plurality of particles comprise poly(N-isopropylacrylamide) (PNIPAM) and wherein the particles are exposed to an elevated temperature.

19. The method of claim 12, wherein the nucleic acid amplification comprises one of rolling circle amplification (RCA), loop-mediated isothermal amplification (LAMP), or polymerase chain reaction (PCR).
